(19) United States
(12) Patent Application Publication
(10) Pub. No.: US 2008/0045756 A1
(43) Pub. Date: Feb. 21, 2008

(54) Process for preparation of a nitrophenol

(75) Inventors: Erie Fache, Caluire (FR); Sebastien Righini, Lyon (FR)

Correspondence Address: Jean-Louis SEUGNET;INTELLECTUAL PROPERTY DEPT., RHODIA INC., 259 PROSPECT PLAINS ROAD, CN-7500,, CRANBURY, NJ, 08512-7500, US

(73) Assignee: RHODIA CHIMIE, AUBERVILLIERS, FRANCE

(21) Appl. No.: 11/898,745

(22) Filed: Sep. 14, 2007

Related U.S. Application Data
(63) Continuation of application No. 10/520,725, filed on Jan. 7, 2005, now abandoned.

(30) Foreign Application Priority Data
Jul. 10, 2002 (FR) 02/08679

(51) Int. Cl. C07C 205/06

(52) U.S. Cl. 568/708

(57) ABSTRACT

The invention relates to a method of preparing a high-purity nitrophenol and, more specifically, p-nitrophenol from a nitrohalobenzene. The inventive method comprises the following steps: (a) hydrolysis of a nitrohalobenzene compound by reacting said compound with a base; (b) acidification in order to produce the nitrophenol compound from the salt thereof by means of an acid treatment; (c) crystallisation of the nitrophenol compound obtained; and (d) separation of the product obtained. The invention is characterised in that it also comprises at least the following steps: (e) concentration of the reaction medium after hydrolysis (a) and before acidification (b); and (f) liquid/liquid decantation after acidification (b) and before crystallisation (c), which is intended to eliminate the water phase obtained after acidification (b).

The object of the present invention is a process for preparation of a nitrophenol of high purity.

More precisely, the invention aims to provide a nitrophenol free from its halogen-containing impurities in inorganic or organic form.

More particularly, the invention concerns a process for preparation of p-nitrophenol.

p-Nitrophenol is an intermediate compound used in the plant protection field, in particular in the preparation of insecticides.

It is also used in the pharmaceutical field, mainly as an intermediate for the production of APAP, namely N-acetyl-p-aminophenol.

For this purpose, the p-nitrophenol must satisfy rather stringent purity requirements.

One of the routes for preparation of p-nitrophenol consists in performing a basic hydrolysis of p-nitrochlorobenzene, most often performed with the use of a solution of sodium hydroxide, then acidification of the sodium phenate salt formed, generally with sulfuric acid.

The problem which arises is that the said process does not result in a p-nitrophenol of high purity, in particular on account of the presence of chlorine, present either in inorganic form, for example owing to the residual presence of p-nitrochlorobenzene, or in organic form (chloride ions), for example owing to the residual presence of sodium chloride which results from the reaction of the p-nitrochlorobenzene and the sodium hydroxide but also on account of the presence of various colored by-products, in particular azo or azoxy species.

In order to mask the formation of colored products, it has been proposed to perform the basic hydrolysis either in the presence of a peroxide (DE 1543952) or by introducing an oxygen-containing gas (U.S. Pat. No. 3,283,011).

Moreover, according to U.S. Pat. No. 3,624,164, a process for preparation of salts of nitrophenols has been described. It consists in performing a basic hydrolysis of p-nitrochlorobenzene followed by concentration of the medium, and crystallization of the corresponding phenate. In addition, the obtention of p-nitrophenol by acidification, followed either by recovery of a liquid p-nitrophenol phase by separation of the aqueous phase, or by recovery of the solid phenol by crystallization of the reaction medium. However, the p-nitrophenol obtained does not meet the desired purity criteria as regards chlorine content.

The objective of the invention is to propose a versatile process which makes it possible to control the purity of the nitrophenol required and to obtain a product meeting the requirements of high purity.

Thus, depending on the choice of steps, it is possible to adjust the purity of the product obtained.

A process has now been found, and it is this that comprises the object of the present invention, for preparation of a nitrophenol from a nitrohalobenzene which consists in performing:
(a) hydrolysis of a nitrohalobenzene compound by reaction of the said compound with a base,
(b) acidification to obtain the nitrophenol compound from its salt, by an acid treatment,
(c) crystallization of the nitrophenol compound obtained,
(d) separation of the product obtained, characterized in that it also includes at least the following steps:
(e) concentration of the reaction medium after hydrolysis (a) and before acidific-ation (b),
(f) liquid/liquid decantation performed after acidification (b) and before crystalliz-ation (c) and intended to remove the aqueous phase obtained after acidification (b).

More precisely, the process comprises the following steps, namely:
(a) hydrolysis of a nitrohalobenzene compound by reaction of the said compound with a base,
(e) concentration of the reaction medium,
(b) acidification to obtain the nitrophenol compound from its salt, by an acid treatment,
(f) liquid/liquid decantation intended to remove the aqueous phase obtained after acidification (b).
(c) crystallization of the nitrophenol compound obtained,
(d) separation of the product obtained.

Thus, according to the invention a nitrophenol is obtained which has a low content of liposoluble impurities (nitrohalobenzenes, for example p-nitrochlorobenzene (PNCB), nitrobenzene (typical impurity of the nitrohalobenzenes) which is shown by:
a nitrohalobenzene (in particular p-nitrochlorobenzene) content less than 180 ppm, preferably less than 50 ppm.

According to the invention, a nitrophenol also freed from impurities soluble in water (sodium chloride, sodium sulfate, hydrophilic organics such as organic sulfonates or sulfates) is obtained, which is shown by:
a halide ions content less than 40 ppm, preferably less than 20 ppm,
a sulfur content preferably less than 200 ppm and still more preferably less 100 ppm.

Thus, the process of the invention also makes it possible to provide a nitrophenol freed from its sulfurous impurities which are present when sulfuric acid is used in the process of the invention as the agent for acidification of the nitrophenol formed intermediately.

The process makes it possible substantially to decrease the sulfur content which is particularly advantageous on account of the intended use of the nitrophenol. In fact, given the fact that the nitro group is reduced to an amino group by catalytic hydrogenation in the course of a subsequent step in order to obtain the APAP, it is desirable that the catalyst contain little residual sulfur, as sulfur is a well-known catalyst poison.

Another typical solution would consist in increasing the number of nitrophenol crystallization operations. However, this alternative is not advantageous in terms of yield and product loss.

According to the process of the invention, which simultaneously combines a concentration and a decantation step, a product which is purer as it has been freed of two types of impurity is obtained.

According to another modification of the invention, an intermediate separation of the nitrophenate obtained following the basic hydrolysis is performed, which makes it possible to obtain a nitrophenol having a still higher degree of purity.

The purity is further improved according to another modification which consists in performing a water washing of the organic phase recovered following the decantation.

A nitrohalobenzene (NHB) is involved in the process of the invention, which can be represented by the following general formula:

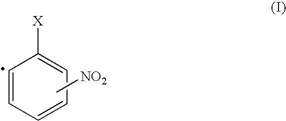

In the said formula (I), X represents a fluorine, chlorine, bromine or iodine atom, preferably a chlorine atom,

In the formula (I), the NO₂ group is in the ortho, meta or para position and preferably in the para position.

The present invention does not exclude the presence of other substituents on the aromatic ring provided that do not interfere with the reactions of the process of the invention. In particular, it is possible that one or several other atom(s) of halogen or of one or several nitro group(s) or of one or several alkyl group(s) having from 1 to 4 carbon atoms may be present.

Several is understood to mean at most 4 substituents.

More particularly, the invention concerns the nitromonohalobenzenes, preferably o-, m- or p-nitrochlorobenzene.

Use may also be made of the product available on the market which is preferably of purity greater than 99%.

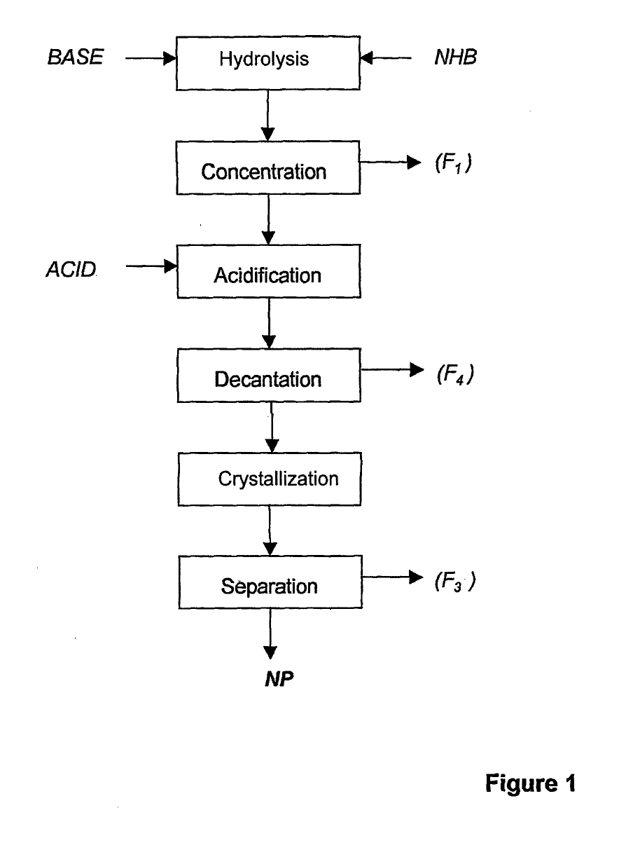

In order to facilitate understanding of the process of the invention, FIGS. 1 to 3 are given below, which present in diagrammatic form the different modifications of the process of the invention, without however restricting the scope of the invention to these.

FIG. 1

According to the process of the invention, firstly the basic hydrolysis of the nitrohalobenzene is performed by reacting it with an inorganic or organic base.

A strong base, that is to say a base having a pKb greater than 12, is preferably chosen, the pKb being defined as the cologarithm of the dissociation of the base, measured in an aqueous medium at 25° C.

Particularly well suited for the implementation of the process of the invention are inorganic bases such as the salts of alkali metals, preferably an alkali metal hydroxide, which can be sodium, cesium, rubidium or potassium hydroxide.

It is possible to use a trialkylammonium hydroxide, but this does not present any additional advantage.

For economic reasons, among all the bases, sodium or potassium hydroxide are preferably chosen.

The concentration of the starting basic solution is not critical. The alkali metal hydroxide solution used is generally of concentration lying between 5 and 70% by weight, preferably 7 to 50%.

The quantity of base introduced into the reaction medium takes account of the quantity necessary to hydrolyze the halogen atom of the starting nitrohalobenzene compound.

Generally, for almost complete or complete conversion of the nitrohalobenzene, the quantity of base expressed by the ratio between the number of moles of base and the number of mole of nitrohalobenzene is at least 2 and preferably lies between 2 and 3. It can however if necessary be less than 2 or greater than 3.

Water is present in the medium, in a quantity such that the nitrohalobenzene represents, in weight percent relative to the totality of the mass of the reaction medium, 1% to 50% and preferably from 7% to 25%.

The process of the invention is advantageously conducted at a temperature lying between 100° C. and 200° C., preferably between 140° C. and 180° C.

The process is preferably conducted under the self-generated pressure of the reactants.

The duration of this hydrolysis step is variable. As an indication, it is stated that it most often takes between 1 and 6 hours. The duration of this step must quite obviously be linked to the other parameters, in particular the temperature.

From a practical point of view, the reactants, nitrohalobenzene and base, can be loaded into an autoclave and then heated. It is also possible to load the reactants consecutively, in particular the sodium hydroxide and then to add the nitrohalobenzene in portions or run it in.

At the end of the hydrolysis reaction, the nitrophenol is obtained in the form of a salt, the associated cation deriving from the base used. Hereinafter it is referred to as “nitrophenate”.

According to one characteristic of the process of the invention, concentration of the reaction medium can be performed so as to increase the concentration of the nitrophenate in the medium from 0.1% by weight to 10% by weight, preferably from 0.5% to 3%.

Thus, in the course of this operation, a fraction (F₁) comprising water (for example from 1 to 20 weight % relative to the mass of the reaction medium coming from the hydrolysis), the starting reactant nitrohalobenzene that has not reacted and all the volatile compounds that can be entrained in steam or can form an azeotrope with water, such as for example nitrobenzene, is eliminated.

By means of this step, the purity of the final product is improved considerably, particularly when the hydrolysis medium contains organic materials of low solubility in water.

A first mode consists simply in decreasing the reaction pressure, by pressure release, while remaining in the aforesaid temperature range. This pressure release is effected in such a manner as to remove at the head the quantity of water necessary to attain the target concentration of nitrophenate in the reaction medium.

By elimination of water and volatile impurities, the desired concentration of nitrophenol, obtained in salt form, is obtained.

Another mode of implementation for concentration of the reaction medium consists in performing the distillation of the quantity of part of the water to attain the desired concentration of nitrophenol, obtained in salt form, in the reaction medium.

The distillation can be performed at atmospheric pressure at a temperature of the order of 100° C.

The distillation can also be performed under a pressure slightly lower than atmospheric pressure, for example from 20 to 750 mm of mercury and at a temperature lower than 100° C. In general, the pressure is chosen so as to have a distillation temperature lying between 80° C. and 99.6° C.

The distillation can also be performed under a pressure greater than atmospheric pressure.

Another mode consists in performing an entrainment by injection of a fluid, for example steam or inert gas, in particular nitrogen.

According to the process of the invention, the acidification of the nitrophenate obtained is performed in a following step in order to generate the hydroxyl group.

For this purpose, the product obtained is firstly brought into aqueous solution or suspension. The quantity of water that may be added is such that the concentration of nitrophenate varies from 10 to 80%, preferably from 15 to 50%.

This operation is performed at a temperature varying between 30° C. and 80° C., preferably between 50° C. and 60° C.

In a following step, the reaction medium is acidified by addition of a protonic acid of inorganic origin, preferably hydrochloric acid or sulfuric acid.

Use is preferably made of a concentrated solution of sulfuric acid of concentration greater than 95% by weight, preferably from 96 to 98%.

The quantity of acid is at least equal to the quantity necessary to neutralize the nitrophenate. Generally, it is such that at the end of acidification a pH lying between 1 and 7, preferably between 2 and 5, is obtained.

The reaction medium is maintained at a temperature varying for example between 45° C. and 70° C., and preferably from 50° C. to 60° C.

The process is preferably conducted under the atmospheric pressure of the reactants.

A two-phase medium is obtained, consisting of a liquid phase comprising a water/nitrophenol mixture essentially containing the nitrophenol (about 70% in the case of p-nitrophenol) and an aqueous phase comprising the excess acid, preferably sulfuric acid, the salts obtained following the acidification, most commonly sodium sulfate and possibly, and in minor amounts, water-soluble organic products, which may for example result from the sulfonation of the benzene nucleus.

According to a preferred modification of the process of the invention, an operation of decantation of the two liquid phases obtained is performed following the acidification, in the range of temperatures stated above for the acidification, preferably 60-70° C.

A separation of the aqueous phase (F₄) comprising the excess sulfuric acid, the salts obtained following the acidification, most commonly sodium sulfate and, in minor amounts, products resulting from the sulfonation of the benzene nucleus of the organic phase comprising essentially nitrophenol, is performed.

Part of this aqueous phase can advantageously be recycled to the hydrolysis or the acidification step. Generally, for example from 10 to 50% of this phase (%) by weight can be recycled.

The solid, essentially consisting of nitrophenol, which precipitates during the cooling of (F₄), can also be recycled to the same steps.

From the organic phase, the crystallization of the nitrophenol is effected by cooling to a temperature which is a temperature lower than 40° C., preferably the ambient temperature or even lower.

During this step, the concentration of the nitrophenol can vary from 10 to 70%, preferably from 20 to 60%.

The separation of the crystallized product is then performed by the standard solid/liquid separation techniques, preferably by filtration or by centrifugation.

The separation is conducted at the temperature of the end of crystallization.

A solid which is essentially the nitrophenol (NP) and an aqueous phase, consisting of the crystallization mother liquors and washing liquors (F₃) containing nitrophenol at the limit of its solubility and saline residues are recovered.

It should be noted that the aqueous phase is of low salinity and can advantageously be recycled to the hydrolysis or phenate acidification step.

One or several washings of the nitrophenol obtained with water can if necessary be effected.

A nitrophenol containing less than 50 ppm of halonitrobenzene but which contains less than 100 ppm of halide and less than 100 ppm of sulfur is recovered.

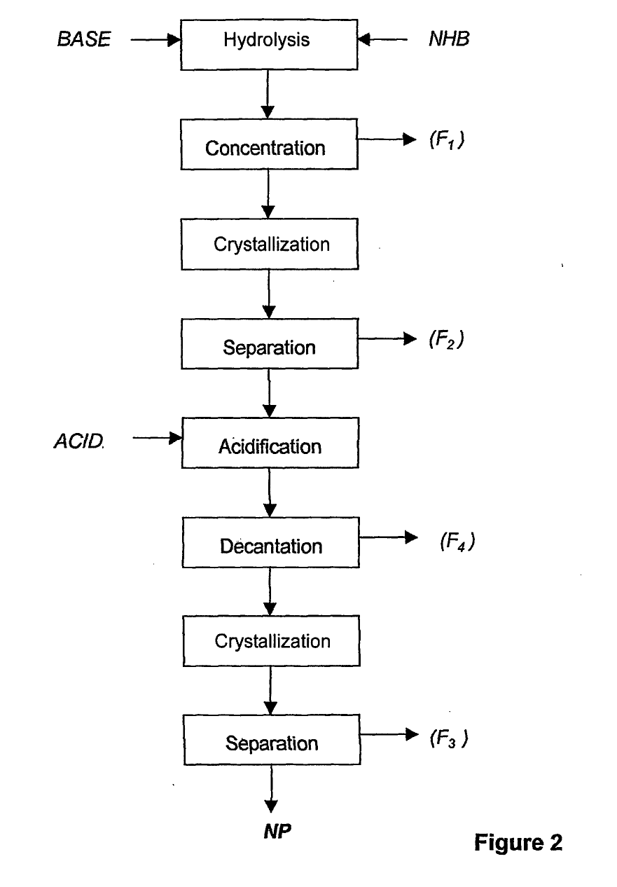

FIG. 2

According to the process of the invention, firstly the basic hydrolysis of the nitrohalobenzene compound is performed as described above and at the end of the reaction the nitrophenol is obtained in salt form.

According to one characteristic of the process of the invention, concentration of the reaction medium can be performed so as to increase the concentration of the nitrophenol in the medium from 0.1% by weight to 10% by weight, preferably from 0.5% to 3%.

Thus, in the course of this operation, a fraction (F₁) comprising water (for example from 1 to 10% weight relative to the mass of the reaction medium coming from the hydrolysis), the starting reactant nitrohalobenzene that has not reacted and all the volatile compounds that can be entrained in steam or can form an azeotrope with water, such as for example nitrobenzene is eliminated.

By means of this step, the desired concentration is obtained and purity of the final product is improved considerably, particularly when the hydrolysis medium contains organic materials of low solubility in water.

To carry out the concentration, the modes of implementation described for FIG. 1 are used.

At the end of this operation, the crystallization of the nitrophenate obtained is performed, by cooling to a temperature which is the ambient temperature (most commonly lying between 15° C. and 25° C.).

The separation of the crystallized product can next be performed by the standard solid/liquid separation techniques, preferably by filtration or centrifugation.

The separation is typically conducted at a temperature lying between 0° C. and 20° C.

A solid which is essentially the nitrophenate and an aqueous phase (F₂) containing the salts generated by the hydrolysis reaction, essentially sodium chloride and a dissolved nitrophenate fraction is recovered.

One or several, for example up to 3, washings can if necessary be performed with water or water saturated with sodium hydroxide or sodium chloride.

According to the process of the invention, the acidification of the nitrophenate obtained is performed in a following step in order to generate the hydroxyl function, which is performed under the conditions described in FIG. 1.

A two-phase medium is obtained, consisting of a liquid phase comprising a water/nitrophenol mixture essentially containing the nitrophenol (about 70% in the case of p-nitrophenol) and an aqueous phase comprising the excess acid, preferably sulfuric acid, the salts obtained following the acidification, most commonly sodium sulfate and possibly, and in minor amounts, water-soluble organic products, which may for example result from the sulfonation of the benzene nucleus.

According to a preferred modification of the process of the invention, an operation of decantation of the two liquid phases obtained is performed following the acidification, in the range of temperatures stated above for the acidification, preferably 60-70° C.

A separation of the aqueous phase (F₄) comprising the excess sulfuric acid, the salts obtained following the acidification, most commonly sodium sulfate and, in minor amounts, products resulting from the sulfonation of the benzene nucleus of the organic phase comprising essentially nitrophenol, is performed.

Part of this aqueous phase can advantageously be recycled to the hydrolysis or the acidification step. Generally, for example from 10 to 50% of this phase (%) by weight can be recycled.

The solid, essentially consisting of nitrophenol, which precipitates during the cooling of (F₄), can also be recycled to the same steps.

From the organic phase, the crystallization of the nitrophenol is effected by cooling to a temperature which is a temperature lower than 40° C., preferably the ambient temperature or even lower.

During this step, the concentration of the nitrophenol can vary from 10 to 70%, preferably from 20 to 60%.

The separation of the crystallized product is then performed by the standard solid/liquid separation techniques, preferably by filtration or by centrifugation.

The separation is conducted at a temperature lying between 0° C. and 20° C.

A solid which is essentially the nitrophenol (NP) and an aqueous phase (F₃) containing nitrophenol at the limit of its solubility and saline residues are recovered.

It should be noted that the aqueous phase is of low salinity and can advantageously be recycled to the nitrophenate aqueous dissolution or suspension step, or indeed also to the hydrolysis.

One or several washings of the nitrophenol obtained with water can if necessary be effected.

A nitrophenol containing less than 50 ppm of halonitrobenzene and less than 50 ppm of halide and less than 100 ppm of sulfur is recovered.

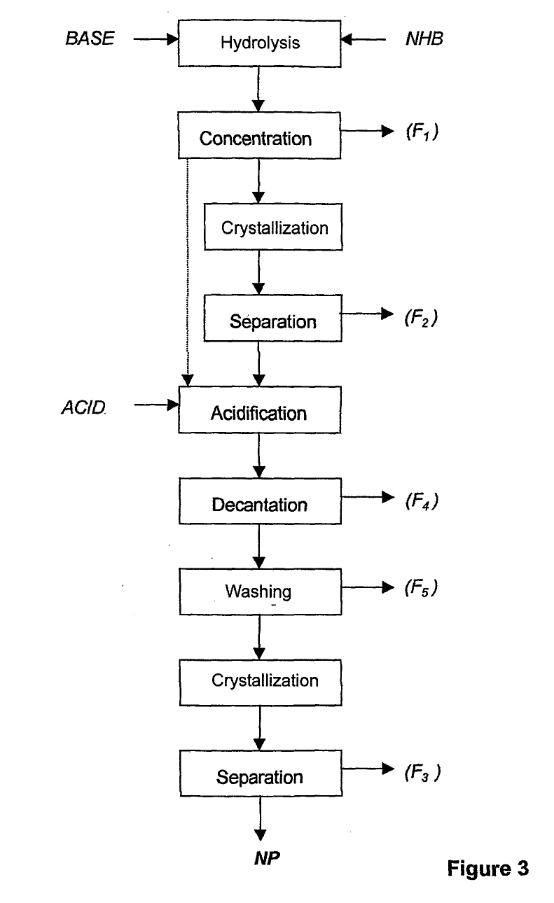

FIG. 3

According to the process of the invention, firstly the basic hydrolysis of the nitrohalobenzene compound is performed as described above and at the end of the reaction the nitrophenol is obtained in salt form.

According to one characteristic of the process of the invention, concentration of the reaction medium can be performed so as to increase the concentration of the nitrophenol in the medium from 0.1% by weight to 10% by weight, preferably from 0.5% to 3%.

Thus, in the course of this operation, a fraction (F₁) comprising water (for example from 1 to 10% weight relative to the mass of the reaction medium coming from the hydrolysis), the starting reactant nitrohalobenzene that has not reacted and all the volatile compounds that can be entrained in steam or can form an azeotrope with water, such as for example nitrobenzene is eliminated.

By means of this step, the desired concentration is obtained and purity of the final product is improved considerably, particularly when the hydrolysis medium contains organic materials of low solubility in water.

To carry out the concentration, the modes of implementation described for FIG. 1 are used.

According to a first modification, the acidification of the nitrophenate obtained is performed in the following step.

According to another modification, crystallization and separation of the nitrophenate obtained is performed immediately before the acidification.

Thus, at the end of the concentration operation, the crystallization of the nitrophenate obtained is performed, by cooling to a temperature which is the ambient temperature (most commonly lying between 15° C. and 25° C.).

The separation of the crystallized product can next be performed by the standard solid/liquid separation techniques, preferably by filtration or centrifugation.

The separation is typically conducted at a temperature lying between 0° C. and 20° C.

A solid which is essentially the nitrophenate and an aqueous phase (F₂) containing the salts generated by the hydrolysis reaction, essentially sodium chloride and a dissolved nitrophenate fraction is recovered.

One or several, for example up to 3, washings can if necessary be performed with water or water saturated with sodium hydroxide or sodium chloride.

According to the process of the invention, the acidification of the nitrophenate obtained is performed in a following step in order to generate the hydroxyl function, which is performed under the conditions described in FIG. 1.

A two-phase medium is obtained, consisting of a liquid phase comprising a water/nitrophenol mixture essentially containing the nitrophenol (about 70% in the case of p-nitrophenol) and an aqueous phase comprising the excess acid, preferably sulfuric acid, the salts obtained following the acidification, most commonly sodium sulfate and possibly, and in minor amounts, water-soluble organic products, which may for example result from the sulfonation of the benzene nucleus.

According to a preferred modification of the process of the invention, an operation of decantation of the two liquid phases obtained is performed following the acidification, in the range of temperatures stated above for the acidification, preferably 60-70° C.

A separation of the aqueous phase (F₄) comprising the excess sulfuric acid, the salts obtained following the acidification, most commonly sodium sulfate and, in minor amounts, products resulting from the sulfonation of the benzene nucleus of the organic phase comprising essentially nitrophenol, is performed.

Part of this aqueous phase can advantageously be recycled to the hydrolysis or the acidification step. Generally, for example from 10 to 50% of this phase (%) by weight can be recycled.

The solid, essentially consisting of nitrophenol, which precipitates during the cooling of (F₄), can also be recycled to the same steps.

In one modification, a step of washing the organic phase is interposed between the decantation and the crystallization.

The quantity of water necessary for this step can vary in large proportions.

By way of indication, it may be stated that the water can be used in a quantity such that there is 0.1 to 2 kg of water per kg of organic phase, preferably from 0.2 to 1 kg of water per kg of organic phase.

Part of the fraction (F₃) derived from a previous production run can be used for this washing step. It should be noted that these new aqueous washings (F₅), which are of low salinity, can advantageously be recycled to the nitrophenate aqueous dissolution or suspension step, or indeed also to the hydrolysis.

From the organic phase, the crystallization of the nitrophenol is effected by cooling to a temperature which is a temperature lower than 40° C., preferably the ambient temperature or even lower.

During this step, the concentration of the nitrophenol can vary from 10 to 70%, preferably from 20 to 60%.

The separation of the crystallized product is then performed by the standard solid/liquid separation techniques, preferably by filtration or by centrifugation.

The separation is conducted at a temperature lying between 0° C. and 20° C.

A solid which is essentially the nitrophenol (NP) and an aqueous phase (F₃) containing nitrophenol at the limit of its solubility and saline residues are recovered.

It should be noted that the aqueous phase is of low salinity and can advantageously be recycled to the nitrophenate aqueous dissolution or suspension step, or indeed also to the hydrolysis.

One or several washings of the nitrophenol obtained with water can if necessary be effected.

A nitrophenol containing less than 50 ppm of halonitrobenzene and less than 20 ppm of halide and less than 100 ppm of sulfur is recovered.

Following the different steps constituting the object of the process of the invention, for example according to the process represented by FIG. 1, a product satisfying all the purity criteria stated at the beginning of the present text is obtained, and according to the processes of FIGS. 2 and 3 a product which can be purer.

Examples of the implementation of the invention are given below.

### EXAMPLES

Before describing the examples in detail, an example is given of the hydrolysis of paranitrochlorobenzene as in the state of the technology.

The synthesis of sodium paranitrophenate (referred to below as phenate) is effected under standard conditions, already previously described (see for example U.S. Pat. No. 3,283,011).

In particular, the reaction masses of hydrolysate, the starting material for our purification sequences, are obtained by heating a reaction mixture consisting of:
PNCB (paranitrochlorobenzene): 1294.5 g (8.2 moles)
sodium hydroxide: 694.4 g (17.4 moles)
water: 4500 g
   for 2 hours in an autoclave at 170° C. (under self-generated pressure of 7-7.2 bars)

After cooling to ambient temperature, the medium is filtered, resulting in a moist solid and mother liquors.

The outcome of the experiment is summarized below.

The p-nitrochlorobenzene is estimated by high performance liquid chromatography (HPLC). The total chlorine and the sulfur are estimated by X-ray fluorescence. The chlorides are estimated by argentimetric titration. The OD (optical density) at 500 nm expresses the coloration of the product, measured by UV/visible spectrometry.

| | | NaPNP | |
|---|---|---|---|
| | Mass (g) | (moles)* | Yield |
| Moist solid S | 2773 | 7.87** | |
| Mother | 3714 | 0.11 | |
| liquors ML | | | |
| Total | 6487 | 7.98 | 97.3% |

HPLC estimation;
**45.3% of phenate expressed as non-hydrated form.

### Example 1

In this example, the p-nitrophenol is prepared by a process utilizing a concentration step and a decantation step.

This example is conducted according to FIG. 1.

352 g of moist solid (S) and 774 g of mother liquors (ML), namely 1.02 moles of phenate, are introduced into a 2 liter multineck flask fitted with a central stirrer (500 rpm) and heated by a double jacket.

The reaction medium is distilled so as to eliminate 74 g of water, under atmospheric pressure.

The reaction medium is brought back to 60° C.

The phenate is then neutralized by acidifying the medium by addition of 56.2 g of concentrated sulfuric acid (96%).

The duration of the addition is 1 hr 30 and the final pH is 3.

At the end of the acidification of the phenate, the aqueous phase (540 g) is removed by decantation.

610 g of water is added to the aqueous phase.

The mixture is cooled to 15° C.

The p-nitrophenol crystallizes.

The solid obtained is filtered.

It is washed with 150 g of deionized water.

After drying, 127 g of dry solid, titrating as 98.5% p-nitrophenol (PNP) (0.904 moles) is obtained.

The analytical results are presented in the summary table (I).

### Example 2

### Effect of Concentration, Crystallization of the Phenate and Decantation

This example is conducted according to FIG. 2.

Example 1 is reproduced, but with the addition of a phenate crystallization step between the concentration step and the phenate acidification step.

After the removal of water to concentrate the medium, it is cooled to 15° C. and the phenate that precipitates is recovered by filtration.

A washing is performed using 160 g of a 13.5% by weight aqueous solution of sodium chloride.

The moist phenate is resuspended in 370 g of water, this is brought up to 60° C., and the phenate is then neutralized by acidifying the medium by addition of 56.5 g of concentrated sulfuric acid (96%).

The decantation is performed at 60° C. and the mixture is cooled to 15° C. to effect the crystallization of the p-nitrophenol and drying is performed as in Example 1.

125.9 g of dry solid, titrating as 99% PNP (0.896 moles), are recovered.

The analytical results are presented in the summary table (I).

### Example 3

### Effect of Concentration, Crystallization of the Phenate, Decantation and Washing of the Organic Phase

This example is conducted according to FIG. 3.

Example 2 is repeated, with the addition of a step of aqueous washing of the decanted organic phase (washing water: 160 g).

124.4 g of dry solid, titrating as more than 99.5% PNP (0.89 moles), are recovered.

The analytical results are presented in the summary table (I).

### Comparison Example 1

In this example, the p-nitrophenol is prepared by a process using neither a concentration step, nor a decantation step.

352 g of moist solid (S) and 774 g of mother liquors (ML), namely 1.02 moles of phenate, are introduced into a 2 liter multineck flask fitted with a central stirrer (500 rpm) and heated by a double jacket.

The reaction medium is brought up to 60° C.

The phenate is then neutralized by acidifying the medium by addition of 56.2 g of concentrated sulfuric acid (96%).

The duration of the addition is 1 hr 30 and the final pH is 3.

The mixture is cooled to 15° C.

The solid obtained is filtered.

It is washed with 132 g of deionized water.

After drying, 140.4 g of dry solid (drying under vacuum for 15 hours at 60° C.), titrating as 96% PNP (0.969 moles) are recovered.

The analytical results are presented in the summary table (I).

### Comparison Example 2

### This Example is Conducted with Omission of the Decantation Step

This example is conducted according to Comparison Example 1 but a concentration step is added between the hydrolysis step and the acidification.

At the end of the hydrolysis, the reaction medium is distilled so as to eliminate 74 g of water.

The analytical results are presented in the summary table (I).

### Comparison Example 3

### This Example is Conducted with Omission of the Concentration Step

This example is conducted according to Comparison Example 1 but a decantation step is added between the acidification of the phenate and the crystallization of the p-nitrophenol.

126 g of dry solid, titrating as 98.4% PNP (0.89 moles), are recovered.

The analytical results are presented in the summary table (I).

### Comparison Example 4

This example is conducted according to Example 2 but with omission of the decantation step which follows the acidification of the phenate.

Following the acidification, the mixture is cooled to 15° C. to effect the crystallization of the p-nitrophenol, and drying is performed as in Example 1.

135.5 g of dry solid, titrating as 96% PNP (0.935 moles), are recovered.

The analytical results are presented in the summary table (I).

TABLE (I)

| | | | | Comparison | Comparison | Comparison | Comparison |
|---|---|---|---|---|---|---|---|
| | Ex. 1 | Ex. 2 | Ex. 3 | Ex. 1 | Ex. 2 | Ex. 3 | Ex. 4 |
| Concentration | Yes | Yes | Yes | No | Yes | No | Yes |
| Crystallization | No | Yes | Yes | No | No | No | Yes |
| of phenate | | | | | | | |
| Hot decantation | Yes | Yes | Yes | No | No | Yes | No |
| Washing of | No | No | Yes | No | No | No | No |
| decanted | | | | | | | |
| organic phase | | | | | | | |
| Purification | 87.9 | 87.7 | 87.1 | 94.8 | 95.0 | 87.2 | 91.5 |
| yield* (%) | | | | | | | |
| Residual PNCB | <50 | <50 | <50 | 180 | <50 | 180 | <50 |
| ppm | | | | | | | |
| Total Cl ppm | 40 | 18 | <10 | 2800 | 2900 | 70 | 300 |
| Chlorides ppm | 20 | <10 | <10 | 2700 | 2750 | 15 | 280 |
| Total S ppm | 30 | 40 | 15 | 26,000 | 25,000 | 40 | 27,000 |
| PNP OD 500 nm | <0.1 | <0.1 | <0.1 | >0.4 | >0.4 | 0.1 | >0.1 |

*PNP/phenate taken

### Example 4

### Recycling of Mother Liquors and Washings from PNP Crystallization and Recycling of the PNP of the Aqueous Decantation and Washing Phases

The example is conducted according to Example 1, except that the crystallization mother liquors and washings derived from the crystallization of the PNP of Example 3 (F₃) are used to dilute the phenate crystallized before acidification: these liquors are concentrated from 850 g to 400 g.

The solid, namely 12 g, which precipitates from the cooled aqueous decantation (F₄) and washing (F₅) phases of Example 3, is recovered.

The precipitate is recycled to the point where the crystallized phenate is diluted.

134 g of dry solid, titrating as more than 99.5% PNP (0.959 moles), are recovered.

The results obtained are as follows:
PNP yield relative to the phenate taken: 95.9%
residual PNCB ppm <50
total Cl ppm <10
total S ppm <50
PNP OD 500 nm <0.1

1-27. (canceled)

28. A process for preparation of a nitrophenol from a nitrohalobenzene comprising the steps of:
(a) hydrolysis of a nitrohalobenzene compound by reaction of the said compound with a base,
(b) acidification to obtain the nitrophenol compound from its salt, by an acid treatment,
(c) crystallization of the nitrophenol compound obtained,
(d) separation of the product obtained,
said process further comprising at least the following steps:
(e) concentration of the reaction medium after hydrolysis (a) and before acidific-ation (b), and
(f) liquid/liquid decantation performed after acidification (b) and before crystalliz-ation (c) in order to remove the aqueous phase obtained after acidification (b).

29- The process according to claim 28, comprising the steps of hydrolysis of the nitrohalobenzene compound, concentration of the reaction medium, acidification, decantation, crystallization of the nitrophenol and separation.

30- The process according to claim 28, comprising the steps of hydrolysis of the nitrohalobenzene compound, concentration of the reaction medium, crystallization of the nitrophenate, separation, acidification, decantation, crystallization of the nitrophenol and separation.

31- The process according to claim 28, comprising the steps of hydrolysis of the nitrohalobenzene compound, concentration of the reaction medium, optionally crystallization of the nitrophenate followed by separation thereof, acidification, decantation, washing of the organic phase, crystallization of the nitrophenol and separation.

32- The process according to claim 28, wherein the basic hydrolysis of the nitrohalobenzene compound is performed by reacting it with an inorganic or organic base.

33- The process according to claim 32, wherein the hydrolysis temperature lies between 100° C. and 200° C.

34- The process according to claim 28, wherein the concentration of the reaction medium is performed so as to increase the concentration of nitrophenol in the medium from 0.1% by weight to 10% by weight.

35- The process according to claim 34, wherein the concentration is increased by decreasing the reaction pressure, by pressure release, while remaining in the aforesaid temperature zone or by distilling under atmospheric pressure at a temperature of the order of 100° C., under a pressure slightly lower than atmospheric pressure selected so as to have a distillation temperature lying between 80° C. and 99.6° C. or at a pressure greater than atmospheric pressure.

36- The process according to claim 28, wherein the acidification is performed by addition of a protonic acid of inorganic origin.

37- The process according to claim 36, wherein the quantity of acid is at least equal to the quantity necessary for obtaining a pH lying between 1 and 7 at the end of acidification.

38- The process according to claim 36, wherein the reaction medium is maintained at a temperature varying between 45° C. and 70°.

39- The process according to claim 28, wherein the crystallization of the nitrophenol is performed by cooling to a temperature which is a temperature lower than 40° C.

40- The process according to claim 28, wherein the separation of the crystallized product is performed by filtration or by centrifugation.

41- The process according to claim 28, wherein a further operation of decantation of the two liquid phases obtained is performed following the acidification, at a temperature of 60-70° C.

42- The process according to claim 30, wherein the crystallization of the nitrophenate is performed at the end of the concentration operation, by cooling to an ambient temperature and that the crystallized product is separated by filtration or centrifugation.

43- The process according to claim 29, wherein a further step of water washing of the organic phase is interposed between the decantation and the crystallization.

44- The process according to claim 43, wherein the mother liquors and washings from the crystallization of the nitrophenol are recycled to the hydrolysis of the nitrohalobenzene or to the dilution of the crystallized phenate after acidification.

45- The process according to claim 43, wherein at least part of the decantation liquors which result the acidification of the nitrophenate is recycled to the hydrolysis of the nitrohalobenzene or to the dilution of the crystallized phenate after acidification.

46- The process according to claim 43, wherein the solid which precipitates to part of the cooled decantation liquors is recycled to the hydrolysis of the nitrohalobenzene or to the dilution of the crystallized phenate after acidification.

47- The process according to claim 31, wherein the aqueous washings of the organic phase which decanted after acidification of the nitrophenate are recycled to the hydrolysis of the nitrohalobenzene or to the dilution of the crystallized phenate after acidification.

48- The process according to claim 28, wherein the nitrohalobenzene corresponds to the formula: 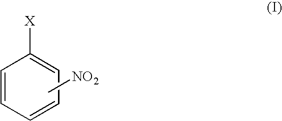
wherein:
X represents a fluorine, chlorine, bromine or iodine atom,
the NO₂ group is in the ortho, meta or para position.

49- The process according to claim 48, wherein the nitrohalobenzene corresponding to the formula (I) bears one or several other halogen atom(s) or one or several nitro group(s) or one or several alkyl group(s) having from 1 to 4 carbon atoms.

50- The process according to claim 48, wherein the nitrohalobenzene is p-nitro-chlorobenzene.

51- A Nitrophenol having:
a nitrohalobenzene content less than 180 ppm, and
a halogen ions content less than 40 ppm.

52- The nitrophenol according to claim 24, having a sulfur content lower than 200 ppm.

53- A p-Nitrophenol having:
a p-nitrohalobenzene content less than 180 ppm, and
a halogen ions content less than 40 ppm.

54- The p-Nitrophenol according to claim 53, wherein it has a sulfur content preferably lower than 200 ppm, and still more preferably lower 100 ppm.
